(19) United States
(12) Patent Application Publication
(10) Pub. No.: US 2023/0089098 A1
(43) Pub. Date: Mar. 23, 2023

(54) CONTACT TRACING USING 5G NETWORK FUNCTIONS

(71) Applicant: International Business Machines Corporation, Armonk, NY (US)

(72) Inventors: Rajesh Kumar Saxena, Maharashtra (IN); ZHENG XIE, Clontarf (IE); Harish Bharti, Pune (IN); Sandeep Sukhija, Rajasthan (IN)

(21) Appl. No.: 17/481,391

(22) Filed: Sep. 22, 2021

(51) Int. Cl. G16H 50/80; G16H 70/60; G16H 50/70; H04W 4/029; H04W 48/16

(52) U.S. Cl. CPC G16H 50/80; G16H 70/60; G16H 50/70; H04W 4/029; H04W 48/16; G16H 10/60

(57) ABSTRACT

A method for contact tracing a confirmed case of an infectious disease. A network function (NF) of a 5G network receives information of a confirmed case of a contagious disease with a consent of the user. The NF accesses mobile device location history and timestamp data of the user having the confirmed case from a unified data repository (UDR) of the 5G network. The NF generates a list of hot spots and timeframes, based on a contact tracing function analyzing the location history, and the NF sends the list of hot spots and timeframes to mobile devices of subscribers of the 5G network, wherein the mobile devices of the 5G subscribers include an application (app) that compares the list of hot spots to locally stored location history on respective mobile devices and provides alerts for a match of location and timeframe.

### BACKGROUND

The present invention relates to location tracing, and more specifically, to contagious disease contact tracing notification using 5G network functions.

Contagious diseases can present a significant threat to the health of human populations, especially diseases that exhibit low barriers to inter-personal transmission. A feature of contagious diseases that significantly facilitates transmission includes airborne transmission, which is very prevalent in areas of high-density population, for example large cities, public transportation, and large populated gatherings, such as sporting events, concerts, and rallies.

An epidemic occurs when there is a widespread occurrence of an infectious disease in a population of people at a particular time. A pandemic occurs when an infectious disease is prevalent over large populations encompassing multiple nations or the entire planet. Infectious diseases result from certain types of bacteria or viruses. Some ways in which infectious diseases are transmitted are by physical contact with surfaces that include disease contamination, by droplets, and by airborne contaminants that result from human coughing, sneezing, singing, shouting, or sometimes just talking or breathing.

Face coverings and interpersonal distancing are often employed to reduce the rapid spread of infectious diseases, such as SARS-2 Covid-19, but people may unknowingly encounter disease contaminants, become infected, and perpetuate transmission. Tracing the historic movement of a user with a confirmed infectious case is useful in determining hot spots in which other users may have been exposed to the infectious disease.

### SUMMARY

According to an embodiment of the present invention, a method, a computer program product, and a system for contact tracing a confirmed case of infectious disease is disclosed. The method for contact tracing a confirmed case of infectious disease provides for one or more processors to receive information of a confirmed case of a first user having a contagious disease, based on an identifier code received with the information with a consent of the user. The one or more processors access location history and timestamp data of a mobile device of the user having the confirmed case from a unified data repository (UDR) of a 5G network. The one or more processors generate a list of hot spots and timeframes associated with respective hot spots, based on a contact tracing function performing analysis of the location history, and the one or more processors send the list of hot spots and the associated timeframes of the respective hot spots to mobile devices of subscribers of the 5G network by using a network function (NF) associated with the subscribers of the 5G network, wherein the mobile devices of the subscribers include an application (app) that compares the locations of the list of hot spots to location history of the respective mobile device, residing on the respective mobile device, and provides alerts for a match of location and timeframe.

### BRIEF DESCRIPTION OF THE DRAWINGS

FIG. 1 is a functional block diagram illustrating a distributed data processing environment, in accordance with an embodiment of the present invention.

FIG. 2 is a flowchart depicting operational steps of a contact tracing program, operating in the network of distributed data processing environment of FIG. 1, in accordance with embodiments of the present invention.

FIG. 3 depicts a block diagram of components of a computing system, including a computing device configured with the capability to operationally perform the contact tracing program of FIG. 2, in accordance with an embodiment of the present invention.

### DETAILED DESCRIPTION

Embodiments of the present invention recognize that controlling the propagation of an infectious disease requires determination of which users are confirmed as infected, controlling further contact between infected users and non-infected users, and tracing the historic locations the confirmed as infected user has occupied during the incubation period of the disease. Determination of contacts that might have been made by the infected user during a period in which the infected user could have transmitted the disease to others involves tracing and timing of infected user movements, often referred to as contact tracing. Embodiments recognize that very manual and tedious processes are involved in contact tracing and that such processes depend a great deal on infected user memory of the locations which the infected user had frequented, and people the infected user may have been in close contact with. Often, in densely populated areas, an infected user would not know those with whom contact was made, creating incomplete traces and ineffective containment.

Embodiments of the present invention recognize that close-spaced enclosed areas with minimal ventilation create ideal conditions for highly pervasive airborne disease transmission. Techniques used to reduce the transmission and exposure of the contagious airborne disease include face coverings, such as filtering masks, and interpersonal distancing. Disease infection can occur in some cases prior to the onset of symptoms indicating the presence of infection (i.e. asymptomatic infection or stage of infection). An infected user, hereafter referred to as a confirmed case, may transmit the disease unknowingly to others contacted by the confirmed case. Contact tracing attempts to determine the locations in which the confirmed case was present to identify and/or notify others that may have been in contact with the confirmed case during an infectious timeframe of the disease and may be at risk of infection as well.

Performing time consuming and resource limited contact tracing has been a primarily manual operation. Manual contract tracing often results in missed opportunities to identify the contacts quickly enough to stop the contagion from becoming widespread. Despite the effort and resource demand, many countries continue to use the manual approach of contact tracing due to the existence of laws protecting personal data. The location history data stored in personal mobile devices and telecom service providers can't be applied to computer-aided analysis for automated contact tracing if the data identifies the user of the mobile device, due to privacy laws, which most likely will increase in protections over time. In a 4G network, for example, to analyze the location data, all the location data of the public individuals would need to be transferred or aggregated to a centralized server, which would violate privacy laws unless explicit consent was documented for each subscriber to the 4G service.

Embodiments of the present invention recognize a need for improved methods of contact tracing that more completely identify locations that may be “hot spots” of potential transmission of the disease and users that may have been in contact with the confirmed case while located in a hot spot. Locations which the confirmed case visited during a period of time equal to the incubation period of the disease when the disease could be transmitted, but the confirmed case user had not yet been confirmed as infected, are referred to, herein, as hot spots. Users in “contact” with the confirmed case at a hot spot location are determined to be within a transmission distance of the confirmed case within a period of time in which the disease contaminant remains infectious and transmittable.

For example, prior to confirming a user as infected, the user takes a cab to a train station and boards a train to a neighboring city to attend a sporting event. Afterward, the user walks to a restaurant and has a meal, returns to the train station via an UBER ride, and travels home. The cab, UBER car, train car, train station, and sporting event areas in which the confirmed case traveled or occupied are considered as hot spots, as are other locations the user visited prior to confirmation of infection. The other users that were in, at, or passed through the hot spots have a risk of acquiring infection from transmission by the confirmed case, and the hot spots need to be identified as well as applying contact tracing to provide notification to the other users. (Note: the term “UBER” may be subject to trademark rights in various jurisdictions throughout the world and are used here only in reference to the products or services properly denominated by the marks to the extent that such trademark rights may exist.)

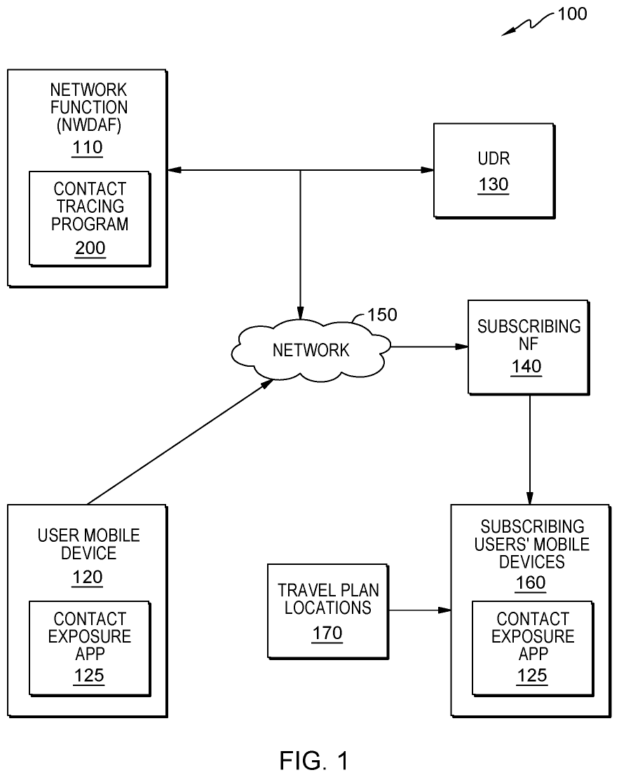

Embodiments of the present invention provide a method, computer program product, and computer system for contact tracing a confirmed case of a user with an infectious disease. Embodiments utilize network functions (NFs) of a 5G network (e.g., Network Data Analytic Function (NWDAF)) to access and analyze location history data of the confirmed case to which the user having the confirmed case has consented. In some embodiments, the installation of the contact exposure app 125 of FIG. 1, on the user's mobile device includes consent by the user to data access for purposes of contact tracing notification. In other embodiments, initiating a triggering message from contact exposure app 125 installed on the mobile device of the user having a confirmed case of a contagious disease establishes consent upon response by the user. A contact tracing model, included in the NWDAF, receives the subscriber information of a Unified Data Repository (UDR) of the 5G network and outputs a list of hot spots and the respective timestamps associated with the hot spots. The contact tracing model (also referred to herein as contact tracing program 200) distributes the hot spot and timestamps to a contact exposure application (app) 125 on the mobile subscribing user's mobile devices 160 of FIG. 1, of the 5G network.

Contact exposure app 125 compares locally stored historic location and timestamp data of the respective subscriber mobile device to the received hot spot and timestamp data and determines if the comparison of location and timeframe indicates an exposure to the user of the respective subscriber mobile device and generates an alert. The resulting contact tracing provides accurate and effective notification to subscribing users and maintains locally stored location history and travel plans.

In some embodiments, a user of a subscribed mobile device that includes the contact exposure app applies future travel route plan locations to compare to received hot spot and timestamp data as a provided preventative feature. The user receives alerts of locations matching the received hotspots that are still within a contagious condition, allowing the user to avoid contact and preventing further transmission of the infectious disease.

Embodiments utilize 5G analytics, at the core, to enable distributed and analytical powered network functions. In some embodiments, the network functions performing the contact tracing are implemented at the network protocol layer to enable access to location data at the transport layer. Implementation at the network protocol layer enables activation globally for situations of rapid geographical transmission, such as in a pandemic. In some embodiments, virtualized distributed network functions perform the contact tracing analysis, which offer high availability, as opposed to the traditional use of centralized servers. Embodiments of the present invention achieve automated contact tracing while maintaining 5G subscriber location history data locally on users' mobile devices in compliance with data privacy laws and policies.

Using 5G analytics, at the 5G core, enables analytic powered network functions (NFs). 5G networks offer performance of contact tracing in virtualized distributed network functions, differing significantly from analysis being traditionally performed on a centralized server. Relays and base stations provide connection of mobile devices to the 5G network. The 5G network includes deployment of the virtualized Network Functions (NFs) and the confirmed case of contagious disease will trigger the NF that the respective mobile phone is connected to. The distributed nature of the NF provides features of robustness and high availability in times when centralized services are prone to fail.

As contact tracing program 200 of FIG. 1, outputs the high probability hot spot locations and timeframes, the distribution of the information relies on propagation to other NFs on other relays and base stations in the network, so other mobile devices and instances of contact exposure app 125 can get notification of the hot spots and generate alerts by comparing the hot spot locations and timeframes to their locally stored location history.

The present invention will now be described with reference to the Figures. FIG. 1 is a functional block diagram illustrating a distributed data processing environment, generally designated 100, in accordance with an embodiment of the present invention. FIG. 1 provides only an illustration of one implementation and does not imply any limitations with regard to the environments in which different embodiments may be implemented. The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Distributed data processing environment 100 includes network function (NF) 110, user mobile device 120, unified data repository (UDR) 130, subscribing network function 140, and subscribing users' mobile devices 160, all interconnected via network 150. In embodiments of the present invention, network 150 is a 5G network-enabled with analytic network functions (NFs), such as a network data analytic function (NWDAF) and subscribing network function 140. Network function 110 includes contact tracing program 200 and first user mobile device 120 and subscribing users' mobile devices 160 include contact exposure app 125. Distributed data processing environment 100 also includes travel plan locations 170.

Embodiments of the present invention implemented on a distributed 5G network include network function 110, which further includes contact tracing program 200. Network function 110 operates as a Network Data Analytic Function (NWDAF) defined by the 3GPP (third generation partnership project) as a standard for using network data analytics in the 5G Core to drive network automation and service orchestration and was introduced to specify the framework for data collection and data analytics in 5G networks. Network Function 110 can receive input from other network functions (NFs), access data from UDR 130, and receive input from application functions (AFs) (not shown).

Network function 110 includes contact tracing program 200 that outputs a listing of hot spot locations, distributed to contact exposure app 125 residing on mobile devices of users subscribing to the 5G network. The hot spot listing includes locations and timeframes in which the confirmed case user visited the respective locations, establishing the potential for exposure and transmission of the contagious disease, for example, Covid-19. In some embodiments, contact tracing program 200 receives information about a confirmed case of contagious disease for a user from user mobile device 120, for example, subsequent to a positive test result or diagnosis. Contact tracing program 200 accesses historic location data and timestamp data associated with the subscription identification (i.e., system identification code) of the mobile device of the confirmed case user that has consented to use of location history, by initiating the triggering information message to the NWDAF. The user's device history includes data that can be accessed through the time series of the contact exposure application. The UDR provides availability of the data to embodiments of the invention, provided user consent has been given.

Contact tracing program 200 performs a contact tracing analysis of the location history by a network function, such as an NWDAF. The network function analyzes the historic location history of the mobile device of the confirmed case user and analyzes the timestamp associated with when the mobile device was at the respective locations. Contact tracing program 200 generates a list of locations visited by the user with the confirmed case of the contagious disease and determines whether the location visited was within the incubation period associated with the contagious disease. Contact tracing program 200 identifies a list of “hot spots”, which are locations in which the potential of exposure and transmission of the contagious disease are high, exceeding a predetermined threshold. Contact tracing program 200 distributes the locations and associated timeframes of hot spots that exceed the threshold level of contagion and transmission risk to contact exposure app 125 operating on subscribing users' mobile devices 160. Contact exposure app 125 compares locally stored location data and timestamp data to the received hot spot and timeframe information determines if there is risk of exposure to the contagious disease by a respective subscribing user. If the comparison of location history and timestamp data indicates a risk of exposure for the respective user, contact exposure app 125 generates an alert on the respective subscribing users' mobile device 160.

User mobile device 120 includes contact exposure app 125 and initiates a triggering message that includes information that the user of user mobile device 120 has a confirmed case of a contagious disease. The information generated from contact exposure app 125 operating on user mobile device 120 is received by network function 110, which, for example, may be a network data analytic function (NWDAF). User mobile device 120 generates location and timestamp data included in a unified data repository (UDR 130) of the 5G network. Access to the location history and timestamp data for contact tracing requires permission by user consent, for example, by installing or initiating contact exposure app 125 on user mobile device 120 a user indicates user consent is granted.

Contact exposure application (app)125 operates on user mobile device 120 and respective subscribing users' mobile devices 160. In an embodiment, a user that has a confirmed case of a contagious disease initiates contact exposure app 125 to generate a message that includes information regarding the confirmed contagious disease. The information may include details of the contagious disease, the subscription ID of the confirmed case user, and information related to determining incubation and acquisition of the contagious disease in the confirmed case user. In embodiments of the present invention, the confirmed case user has provided consent to provide the 5G subscription ID of the user.

In embodiments of the present invention, contact exposure app 125 also performs a comparison between location history and timestamps held locally on respective subscribing users' mobile devices 160 and the received location and timeframe information indicating hot spots exceeding a threshold level of transmission of the contagious disease. Contact exposure app 125 determines whether a respective user had potential exposure to the contagious disease and indicates potential exposure for future travel plans of respective users made available to contact exposure app 125 on the respective subscribing users' mobile devices 160.

UDR 130 include 5G subscriber information such as information associated with subscribers' profile, service or subscription identification, applications, authentication, service authorization, policy data, session binding, and Application state information. In some embodiments, a subscription profile includes information regarding user registration, data network profile, and authorizations. In some embodiment implementations, the dataset associated with a subscribed device may be redacted as part of the user plane separation resulting from the control and user plane separation (CUPS), ensuring that the user data is used to access the location history of the confirmed case user. In some embodiments, UDR 130 stores subscriber location history and timestamp information, and subsequent to receiving a triggering message with consent by the user having a confirmed case of the contagious disease, network function 110 accesses the location history and timestamp data of the confirmed case user from UDR 130.

Subscribing NF 140 receives output from network function 110, such as an NWDAF, and performs distribution of the output to subscribing users' mobile devices 160. The 5G network includes a plurality of network functions, such as subscribing NF 140 with each performing a designated action or operation with respect to the 5G network. In some embodiments of the present invention, subscribing NF 140 includes or accesses information to enable distribution of hot spot locations and associated timeframes, received from network function 110, which exceed a predetermined threshold of exposure risk to the contagious disease of a confirmed case of a user. In some embodiments, subscribing NF 140 represents a plurality of subscribing network functions on the 5G network.

Subscribing users' mobile devices 160 represents a plurality of 5G network subscribed user mobile devices. The 5G network (network 150) includes network function 110, UDR 130, and subscribing NF 140 as connected members. Subscribing users' mobile devices 160 can perform communication operations, download and operate applications, and perform other actions and functions on or independent of network 150. Subscribing users' mobile devices 160 includes, respectively, contact exposure app 125 described above, that performs an alert to the respective user in response to determining an exposure risk to a contagious disease, based on locally stored location history and timestamp data associated with the respective mobile device.

Travel plan locations 170 includes a set of stored data on respective mobile devices of subscribing users' mobile devices 160. The stored data includes locations of future travel plans for a respective user. In some embodiments, contact exposure app 125 accesses travel plan locations 170 and determines whether future planned travel aligns with hot spot locations received from contact tracing program 200 via subscribing NF 140, thus offering prevention of exposure to potentially contaminated and disease transmissible locations.

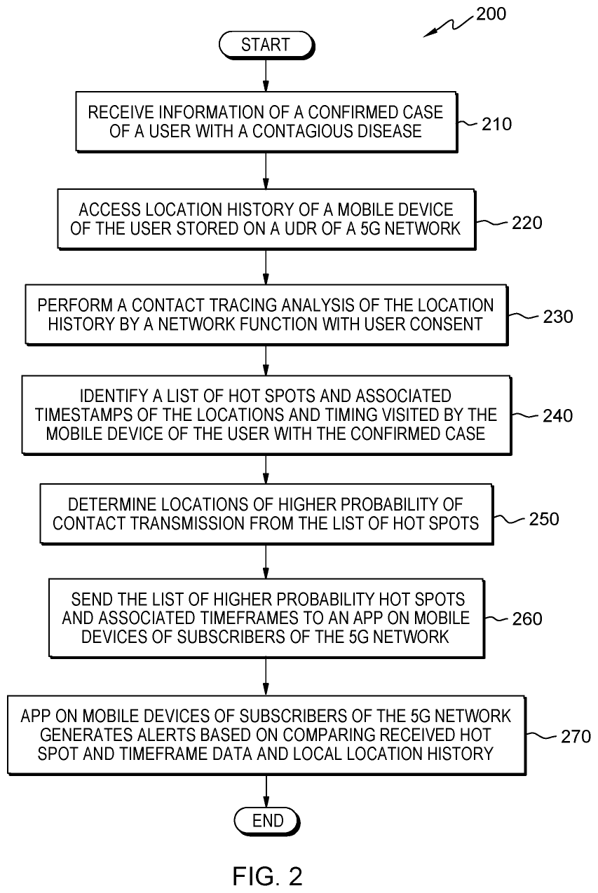

FIG. 2 illustrates operational steps of contact tracing program 200, operating on network function 110, within distributed data processing environment 100 of FIG. 1, in accordance with an embodiment of the present invention.

Contact tracing program 200 receives information of a confirmed case of a user with a contagious disease (step 210). A contact exposure app operating on a mobile device of the user with the confirmed case initiates a message containing the information. The received message triggers contact tracing program 200, which operates integrally to a network function, such as a Network Data Analytic Function (NWDAF) on a 5G network. The received information indicates the type of contagious disease and known stages indicating the state of the infection regarding transmission and infection susceptibility. The received information indicates a subscription identification (SID) associated with the confirmed case user's mobile device, which is used to determine location history and timestamp data associated with the confirmed case user's mobile device. In embodiments of the present invention, the user with the confirmed case of contagious disease provides consent for access and use of location history and timestamp data of the user's mobile device.

For example, a message is initiated from contact exposure app 125 operating on user mobile device 120, indicating consent has been given by the user to access location history and timestamp data of user mobile device 120. Contact tracing program 200, operating on network function 110, receives the message and includes the subscription identification code of user mobile device 120.

Contact tracing program 200 accesses the location history and timestamp data of the mobile device of the confirmed case user stored on a unified data repository of the 5G network (step 220). Contact tracing program 200 accesses the location history and timestamp data from the UDR of the 5G network, based on the SID in the received message information associated with the confirmed case user. In some embodiments, the received information includes the starting point of location history to access, such as the type of contagious disease and the incubation period associated with the disease, as well as information related to a state of the infection of the confirmed case user. The location history data acquired by contact tracing program 200 includes a timestamp of when the mobile device of the confirmed case user was at the location and the duration of stay at the location. In some embodiments, contact tracing program 200 determines whether the location is an indoor or outdoor location, based on additional network data access from additional network functions (not shown in FIG. 1).

Contact tracing program 200 performs a contact tracing analysis of the location history (step 230). Contact tracing program 200 operates as integral to a network function, such as an NWDAF. Contact tracing program 200 analyzes the location history and the timestamps associated with each respective location and determines a risk of contamination and transmission exposure for the locations, which also includes a time range in which effective contamination and transmission potential exist. In some embodiments, the historic location data includes global positioning unit (GPU) coordinates, and in some embodiments, known location structures or features corresponding to GPU coordinates are identified, such as buildings, parks, public transportation, etc.

Contact tracing program 200 identifies a list of hot spots and associated timestamps of the locations and timing visited by the mobile device of the user with the confirmed case (step 240). Having performed analysis of the location history and timestamp data, contact tracing program 200 identifies “hot spot” locations, based on identifying the locations, determining the timing of being at the location relative to the timing and incubation period of the contagious disease, and the duration of stay of the mobile device at the respective locations. In some embodiments, a “hot spot” may be a specific location point, a route taken by the confirmed case user, a unit of public transportation, an assembly or gathering location, or locations in which interaction or close proximity with other users occurs.

Contact tracing program 200 determines locations of higher probability of contact transmission from the list of hot spots (step 250). Subsequent to identifying hot spots from the location history, contact tracing program 200 determines locations with a higher probability of transmission of the contagious disease. Determination of a higher probability of transmission includes analysis of the time range component of the location history as well as considering the type of contagious disease, the type of location (i.e., indoors/outdoors), the density of users at the location, duration of stay, cleaning practices, and other factors at the location, if known. For example, certain contagious diseases may have effective physical contact transmission from touching contaminated objects lasting for several days. Locations in which users commonly touch objects, such as railings, doorknobs, and subway handles may present a hot spot location for several days. Similarly, airborne contagious diseases may be more transmissible in indoor or enclosed locations with confined spacing and contamination may linger in unventilated air for hours. Contact tracing program 200 determines the hot spots that present a higher probability of contact transmission from the original list of hot spots. In some embodiments, contact tracing program 200 includes a predetermined threshold to identify higher probability contact transmission locations, based on assigning weights to known conditions that affect the transmission of the contagious disease. Examples of factors receiving weighted values include indoor or outdoor locations, duration of stay of the confirmed case user at a location, known cleaning or ventilation conditions for the location, etc.

Contact tracing program 200 sends the list of high probability hot spots and associated timeframes to an app on the mobile devices of subscribers of the 5G network (step 260). Contact tracing program 200 sends the list of high probability hot spots and associated timeframes to mobile devices of subscribing users of the 5G network. In some embodiments, contact tracing program 200 sends the list of higher probability hot spots to network functions of the 5G network associated with subscribing users of the network, which in turn distributes the list of higher probability hot spots to the subscribers' mobile devices.

Having sent the list of higher probability hot spots to subscribing users' mobile devices via additional network functions of the 5G network, contact exposure app 125, operating on the mobile devices of the subscribing users, compares the received hot spot location and timeframe windows of exposure to the location history stored locally on respective subscribing users' mobile devices (illustrated as step 270 of FIG. 2). Contact exposure app 125 generates an alert indicating exposure to the contagious disease upon determining a comparison match of location and timing. A feature of embodiments of the present invention is providing contact tracing notification and alerts while maintaining location history and timeframe information on the local mobile device of a user. In some embodiments, contact exposure app 125 provides recommended actions for a user of the mobile device that compares the received hot spot location and timeframe windows of exposure to the location history stored locally on the subscribing user's mobile device, based on the information associated with the contagious disease and a degree of exposure.

For example, contact exposure app 125 operating on a respective subscribing users' mobile device 160 receives a list of a high probability of transmission hot spots from contact tracing program 200, operating on network function 110 of 5G network 150, via subscribing NF 140. Contact exposure app 125 determines whether locally stored location history and timestamp data match one or more of the received high probability of transmission hot spot data. In response to determining a match between location and timeframe, contact exposure app 125 generates an alert to the user of the mobile device providing the information associated with the potential transmission exposure.

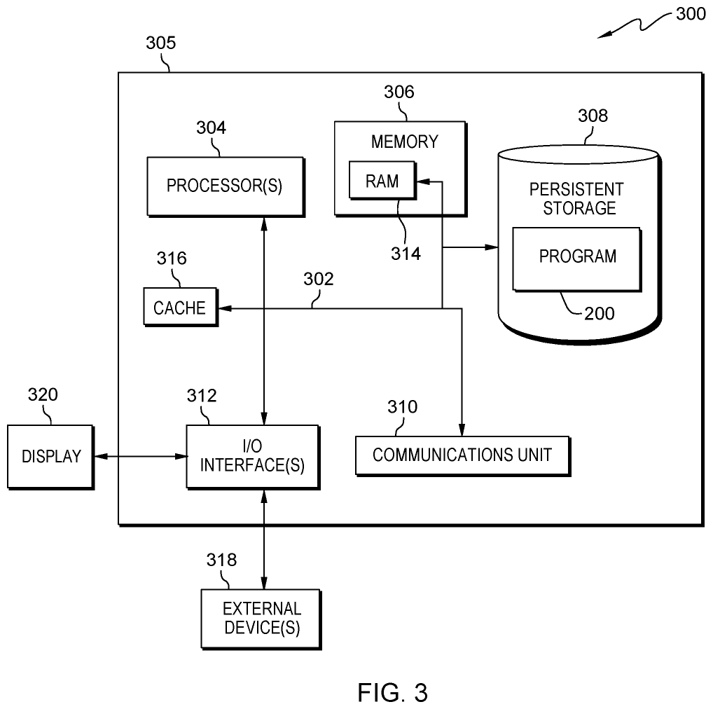

FIG. 3 depicts a block diagram of components of system 300, which includes computing device 305. Computing device 305 includes components and functional capability similar to network function 110, user mobile device 120, and subscribing user's mobile devices 160 (FIG. 1), in accordance with an illustrative embodiment of the present invention. It should be appreciated that FIG. 3 provides only an illustration of one implementation and does not imply any limitations with regard to the environments in which different embodiments may be implemented. Many modifications to the depicted environment may be made.

Computing device 305 includes communications fabric 302, which provides communications between computer processor(s) 304, memory 306, persistent storage 308, communications unit 310, and input/output (I/O) interface(s) 312. Communications fabric 302 can be implemented with any architecture designed for passing data and/or control information between processors (such as microprocessors, communications and network processors, etc.), system memory, peripheral devices, and any other hardware components within a system. For example, communications fabric 302 can be implemented with one or more buses.

Memory 306, cache memory 316, and persistent storage 308 are computer-readable storage media. In this embodiment, memory 306 includes random access memory (RAM) 314. In general, memory 306 can include any suitable volatile or non-volatile computer-readable storage media.

Contact tracing program 200 is stored in persistent storage 308 for execution by one or more of the respective computer processors 304 via one or more memories of memory 306. In this embodiment, persistent storage 308 includes a magnetic hard disk drive. Alternatively, or in addition to a magnetic hard disk drive, persistent storage 308 can include a solid-state hard drive, a semiconductor storage device, read-only memory (ROM), erasable programmable read-only memory (EPROM), flash memory, or any other computer-readable storage media that is capable of storing program instructions or digital information.

The media used by persistent storage 308 may also be removable. For example, a removable hard drive may be used for persistent storage 308. Other examples include optical and magnetic disks, thumb drives, and smart cards that are inserted into a drive for transfer onto another computer-readable storage medium that is also part of persistent storage 308.

Communications unit 310, in these examples, provides for communications with other data processing systems or devices, including resources of distributed data processing environment 100, such as network function 110, user mobile device 120, UDR 130, subscribing NF 140, and subscribing users' mobile devices 160. In these examples, communications unit 310 includes one or more network interface cards. Communications unit 310 may provide communications through the use of either or both physical and wireless communications links. Contact tracing program 200 may be downloaded to persistent storage 308 through communications unit 310.

I/O interface(s) 312 allows for input and output of data with other devices that may be connected to computing system 300. For example, I/O interface 312 may provide a connection to external devices 318 such as a keyboard, keypad, a touch screen, and/or some other suitable input device. External devices 318 can also include portable computer-readable storage media such as, for example, thumb drives, portable optical or magnetic disks, and memory cards. Software and data used to practice embodiments of the present invention, e.g., contact tracing program 200, can be stored on such portable computer-readable storage media and can be loaded onto persistent storage 308 via I/O interface(s) 312. I/O interface(s) 312 also connects to a display 320.

Display 320 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus, or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for contact tracing a confirmed case of an infectious disease, the method comprising:
receiving, by one or more processors of a network function, information of a confirmed case of a first user having a contagious disease, based on a subscriber identification code received with the information;
accessing, by the one or more processors of the network function, a first location history and associated timestamps of a first mobile device of the first user having the confirmed case from a unified data repository (UDR) of a 5G network;
generating, by the one or more processors of the network function, a list of hot spots and timeframes associated with respective hot spots of the list of hot spots, based on a contact tracing function performing analysis of the first location history; and
sending, by the one or more processors of the network function, the list of hot spots and the associated timeframes associated with the respective hot spots of the list of hot spots to mobile devices of subscribers of the 5G network.

2. The method of claim 1, wherein the network function is a network data analytic function (NWDAF).

3. The method of claim 1, wherein generating the list of hot spots and timeframes associated with the respective hot spots of the list of hot spots is performed by a contact tracing model integrated with the network function of the 5G network and includes consent given by users subscribing to the 5G network.

4. The method of claim 1, further comprising:
determining, by the one or more processors of the network function, a set of hot spots having a high probability of transmission of the contagious disease, taken from the list of hot spots and timeframes associated with the respective hot spots of the list of hot spots, based on information associated with a type of contagious disease of the confirmed case, and conditions associated with locations of the first location history.

5. The method of claim 1, wherein the method is implemented at a network protocol layer of the 5G network.

6. The method of claim 1, wherein the analysis of the first location history and the associated timestamps of the first mobile device of the first user is done in virtualized distributed network functions of the 5G network.

7. The method of claim 1, further comprising:
assigning, by the one or more processors, a weighted value to hot spot locations determined from the first location history and associated timestamps of the first mobile device of the first user; and
determining, by the one or more processors, a threshold for hot spots of high probability of transmission of the contagious disease from the list of hot spots and timeframes associated with respective hot spots, based on the weighted value assigned to hot spot locations of the location history of the mobile device of the first user.

8. The method of claim 1, further comprising:
receiving, by one or more processors of a second mobile device, a list including hot spot locations and timeframes associated with the hot spot locations, wherein the hot spot locations are identified as locations with a time range of exposure to a confirmed case of a contagious disease and the hot spot locations and the timeframes associated with the hot spot locations are received from a network function of the 5G network;
comparing, by the one or more processors of the second mobile device, the list of hot spot locations and timeframes to a second location history and timestamp associated with the location history stored on the second mobile device; and
responsive to determining a match between the list of hot spot locations and timeframes and the second location history and timestamps stored on the second mobile device, generating, by the one or more processors of the second mobile device, an alert of exposure to the confirmed case of the infectious disease;
displaying, by one or more processors of the second mobile device, the alert of the exposure to the confirmed case of the infectious disease to a user of the second mobile device; and
providing, by the one or more processors of the second mobile device, recommended actions for the user of the second mobile device that compares the received hot spot location and the timeframes of exposure to the second location history stored locally on the second mobile device, based on information associated with the contagious disease and a degree of exposure of the user to the contagious disease.

9. A computer program product for contact tracing a confirmed case of an infectious disease, the method comprising:
one or more computer readable storage media, and program instructions collectively stored on the one or more computer readable storage media, the program instructions comprising:
program instructions to receive information of a confirmed case of a first user having a contagious disease, based on a subscriber identification code received with the information;
program instructions to access location history and associated timestamps of a mobile device of the first user having the confirmed case from a unified data repository (UDR) of a 5G network;
program instructions to generate a list of hot spots and timeframes associated with respective hot spots of the list of hot spots, based on a contact tracing function performing analysis of the location history; and
program instructions to send the list of hot spots and the associated timeframes associated with the respective hot spots of the list of hot spots to mobile devices of subscribers of the 5G network.

10. The computer program product of claim 9, wherein program instructions to generate the list of hot spots and timeframes associated with the respective hot spots of the list of hot spots is performed by a contact tracing model integrated with the network function of the 5G network and includes consent given by users subscribing to the 5G network.

11. The computer program product of claim 9, further comprising:
program instructions to determine a set of hot spots having a high probability of transmission of the contagious disease, taken from the list of hot spots and timeframes associated with the respective hot spots of the list of hot spots, based on information associated with a type of contagious disease of the confirmed case, and conditions associated with locations of the location history.

12. The computer program product of claim 9, wherein program instructions for the analysis of the location history and the associated timestamps of the mobile device of the first user are done in virtualized distributed network functions of the 5G network.

13. The computer program product of claim 9, further comprising:
program instructions to assign a weighted value to hot spot locations determined from the location history and associated timestamps of the mobile device of the confirmed case user; and
program instructions to determine a threshold for hot spots of high probability of transmission of the contagious disease from the list of hot spots and timeframes associated with respective hot spots, based on the weighted value assigned to hot spot locations of the location history of the mobile device of the confirmed case user.

14. The computer program product of claim 9, further comprising:
program instructions for a mobile device of a subscribing user of the 5G network to receive a list including hot spot locations and timeframes associated with the hot spot locations, wherein the hot spot locations are identified as locations with a time range of exposure to a confirmed case of a contagious disease and the hot spot locations and the timeframes associated with the hot spot locations are received from a network function of the 5G network;
program instructions for a mobile device of a subscribing user of the 5G network to compare the list of hot spot locations and timeframes to a second location history and timestamp associated with the location history stored on the second mobile device; and
responsive to determining a match between the list of hot spot locations and timeframes and the second location history and timestamps stored on the second mobile device, program instructions for a mobile device of a subscribing user of the 5G network to generate an alert of exposure to the confirmed case of the infectious disease;
program instructions for a mobile device of a subscribing user of the 5G network to display the alert of the exposure to the confirmed case of the infectious disease to a user of the second mobile device; and
program instructions for a mobile device of a subscribing user of the 5G network to provide recommended actions for the user of the second mobile device that compares the received hot spot location and the timeframes of exposure to the second location history stored locally on the second mobile device, based on information associated with the contagious disease and a degree of exposure of the user to the contagious disease.

15. A computer system for contact tracing a confirmed case of an infectious disease, the computer system comprising:
one or more computer processors;
one or more computer-readable storage media, and program instructions stored on the one or more computer readable storage media for execution by at least one of the one or more computer processors, the program instructions comprising:
program instructions to receive information of a confirmed case of a first user having a contagious disease, based on a subscriber identification code received with the information;
program instructions to access location history and associated timestamps of a mobile device of the first user having the confirmed case from a unified data repository (UDR) of a 5G network;
program instructions to generate a list of hot spots and timeframes associated with respective hot spots of the list of hot spots, based on a contact tracing function performing analysis of the location history; and
program instructions to send the list of hot spots and the associated timeframes associated with the respective hot spots of the list of hot spots to mobile devices of subscribers of the 5G.

16. The computer system of claim 15, wherein the network function is a network data analytic function (NWDAF).

17. The computer system of claim 15, wherein program instructions to generate the list of hot spots and timeframes associated with the respective hot spots of the list of hot spots is performed by a contact tracing model integrated with the network function of the 5G network.

18. The computer system of claim 15, further comprising:
program instructions to determine a set of hot spots having a high probability of transmission of the contagious disease, taken from the list of hot spots and timeframes associated with the respective hot spots of the list of hot spots, based on information associated with a type of contagious disease of the confirmed case, and conditions associated with locations of the location history.

19. The computer system of claim 15, wherein program instructions for the analysis of the location history and the associated timestamps of the mobile device of the first user are performed by virtualized distributed network functions of the 5G network.

20. The computer system of claim 15, further comprising:
program instructions to assign a weighted value to hot spot locations determined from the location history and associated timestamps of the mobile device of the confirmed case user; and
program instructions to determine a threshold for hot spots of high probability of transmission of the contagious disease from the list of hot spots and timeframes associated with respective hot spots, based on the weighted value assigned to hot spot locations of the location history of the mobile device of the confirmed case user.
